(19) United States
(12) Patent Application Publication
(10) Pub. No.: US 2017/0100314 A1
(43) Pub. Date: Apr. 13, 2017

(54) THICKENED SKIN CARE PRODUCT

(71) Applicant: Henkel AG & Co. KGaA, Duesseldorf (DE)

(72) Inventors: Susanne Dickhof, Viersen (DE); Marianne Waldmann-Laue, Monheim (DE); Soraya Heinen, Koeln (DE); Ira Hartmann, Duesseldorf (DE)

(73) Assignee: Henkel AG & Co. KGaA, Duesseldorf (DE)

(21) Appl. No.: 15/288,053

(22) Filed: Oct. 7, 2016

(30) Foreign Application Priority Data
Oct. 12, 2015 (DE) 10 2015 219 712.3

(51) Int. Cl. A61K 8/37; A61Q 19/02; A61K 8/31; A61Q 17/04; A61K 8/06; A61K 8/34

(52) U.S. Cl. CPC A61K 8/375; A61K 8/062; A61K 8/342; A61K 8/31; A61Q 17/04; A61Q 19/02; A61K 2800/48

(57) ABSTRACT

Cosmetic compositions in the form of an oil-in-water emulsion, including:
a) at least one monoglycerol fatty acid ester A,
b) at least one first polyglycerol di-fatty acid ester B,
c) at least one second polyglycerol di-fatty acid ester C different from the first polyglycerol di-fatty acid ester B,
d) at least one fatty acid ester of the general formula (I)

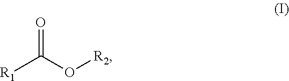

where the R₁ and R₂ groups independently of one another stand for a saturated or unsaturated alkyl groups having 16 to 26 carbon atoms,
e) at least one thickener, and
f) at least one cosmetically active substance.
   Methods are included for using these compositions.

### FIELD OF THE INVENTION

The present invention generally relates to a cosmetic composition for skin care based on a specific emulsifier combination, the use of such a specific emulsifier combination for improving the temperature stability of cosmetic compositions, and a method for skin care in which a cosmetic composition is applied to the skin, in particular to facial skin.

### BACKGROUND OF THE INVENTION

The human skin as man's largest organ performs a number of vital functions. Having an average area of about 2 m² in adults, it has a prominent role as a protective and sensory organ. The purpose of this organ is to transmit and avert mechanical, thermal, actinic, chemical, and biological stimuli. In addition, it has an important role as a regulatory and target organ in human metabolism. The cosmetic treatment of the skin therefore is an important part of human body care. The aim of skin care in the cosmetic sense is to strengthen or rebuild the skin's natural function as a barrier against weather and environmental influences and against the loss of endogenous substances (e.g., water, natural fats, electrolytes), and to slow the natural aging process in order to give the skin a young and healthy appearance.

Therefore, cosmetic active substances for the treatment of the skin that impart care properties, properties that protect from signs of aging, or revitalizing properties are of particular interest. Preparations for skin care and for skin protection, including a whole range of different cosmetic active substances or active substance complexes, are offered in great quantities and in many preparation forms.

In order to increase the cosmetic effectiveness of these preparations, they include the cosmetically active substances preferably in a mixture and/or in a high concentration.

The physical stabilization of complex or highly concentrated active substance systems has proven to be difficult, however, particularly in aqueous sera and gels. This technical difficulty can be countered in fact with the use of increased amounts of thickeners or emulsifiers, but as a result, cosmetic parameters such as skin feel, absorption, and tackiness of the cosmetic composition are detrimentally influenced.

It is therefore desirable to provide a basic cosmetic formulation that is suitable for cosmetic compositions with a high active substance content and low emulsifier content. The cosmetic compositions should be characterized by good cosmetic compatibility, good applicability, and low tackiness. The advantageous cosmetic properties of the composition and its physical stability should be retained in this case over a long time period as well and also under rigid conditions (e.g., long storage at elevated temperatures).

The German patent application DE 19919630 A1 describes sunscreen products that include, in addition to other components, polyglycerol fatty acid esters and organic UV filters.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic composition in the form of an oil-in-water emulsion includes at least one monoglycerol fatty acid ester A, at least one first polyglycerol di-fatty acid ester B, at least one second polyglycerol di-fatty acid ester C different from the first polyglycerol di-fatty acid ester B, at least one fatty acid ester of the general formula (I)

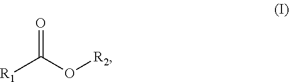

where the R₁ and R₂ groups independently of one another stand for a saturated or unsaturated alkyl groups having 16 to 26 carbon atoms, at least one thickener, and at least one cosmetically active substance.

A composition that includes at least one monoglycerol fatty acid ester A, at least one first polyglycerol di-fatty acid ester B, at least one second polyglycerol di-fatty acid ester C different from the first polyglycerol di-fatty acid ester, at least one fatty acid ester of the general formula (I)

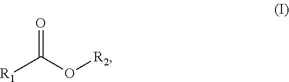

where the R₁ and R₂ groups independently of one another stand for a saturated or unsaturated alkyl groups having 16 to 26 carbon atoms, is used to improve the storage stability of a cosmetic composition including at least one UV filter; to improve the UV protective effect of a cosmetic composition including at least one UV filter; to improve the absorption of a cosmetic composition including at least one UV filter; and to improve the spreadability and to reduce the tackiness of a cosmetic composition including at least one UV filter.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was now found surprisingly that compositions that include, in addition to a combination of four specific fatty acid esters, furthermore at least one thickener, achieve the aforesaid objects. Cosmetic compositions based on this combination are distinguished, compared with conventional compositions of the prior art, by good cosmetic compatibility, good applicability, and low tackiness and, moreover, are physically and chemically stable also at elevated temperatures for a long time period.

Physical stability in this case means the stability of the rheological properties of the cosmetic composition and its stability against phase separation including stability against sedimentation of suspended solids.

The term ‘chemical stability’ describes the stability against chemical transformation or degradation as can be tested, for example, by an activity test of the cosmetically active substances d).

The following are provided by the present invention:

1. A cosmetic composition in the form of an oil-in-water emulsion, including:
a) at least one monoglycerol fatty acid ester A,
b) at least one first polyglycerol di-fatty acid ester B,
c) at least one second polyglycerol di-fatty acid ester C different from the first polyglycerol di-fatty acid ester B,
d) at least one fatty acid ester of the general formula (I)

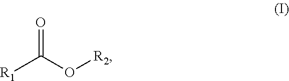

where the R₁ and R₂ groups independently of one another stand for a saturated or unsaturated alkyl groups having 16 to 26 carbon atoms,
e) at least one thickener,
f) at least one cosmetically active substance.

2. The cosmetic composition according to point 1, wherein the monoglycerol fatty acid ester A is selected from the group of mono-, di-, or triesters of glycerol with behenic acid.

3. The cosmetic composition according to one of the preceding points, wherein the monoglycerol fatty acid ester A is selected from the group of compounds with the INCI name Glyceryl Behenate.

4. The cosmetic composition according to one of the preceding points, wherein the monoglycerol fatty acid ester A, based on the total weight of the compositions, is included in amounts of 0.01 to 6.0% by weight, preferably in amounts of 0.1 to 4.0% by weight, and in particular in amounts of 0.5 to 2.0% by weight.

5. The cosmetic composition according to one of the preceding points, wherein the first polyglycerol di-fatty acid ester B is selected from the group of di-, tri-, or tetraglycerol di-fatty acid esters, preferably from the group of triglycerol di-fatty acid esters.

6. The cosmetic composition according to one of the preceding points, wherein the first polyglycerol di-fatty acid ester B is selected from the group of triglycerol di-fatty acid esters of saturated or unsaturated fatty acids having 16 to 30 carbon atoms, preferably from the group of triglycerol di-fatty acid esters of fatty acids from the group comprising palmitic acid, palmitoleic acid, oleic acid, cerotic acid, and melissic acid.

7. The cosmetic composition according to one of the preceding points, wherein the first polyglycerol di-fatty acid ester B is selected from the group of compounds with the INCI name PEG-3 Beeswax.

8. The cosmetic composition according to one of the preceding points, wherein the first polyglycerol di-fatty acid ester B, based on the total weight of the compositions, is included in amounts of 0.05 to 3.0% by weight, preferably in amounts of 0.1 to 2.0% by weight, and in particular in amounts of 0.2 to 1.5% by weight.

9. The cosmetic composition according to one of the preceding points, wherein the second polyglycerol di-fatty acid ester C is selected from the group of penta-, hexa-, or heptaglycerol di-fatty acid esters, preferably from the group of hexaglycerol di-fatty acid esters.

10. The cosmetic composition according to one of the preceding points, wherein the second polyglycerol di-fatty acid ester C is selected from the group of hexaglycerol di-fatty acid esters of saturated or unsaturated fatty acids having 16 to 30 carbon atoms, preferably from the group of hexaglycerol di-fatty acid esters of saturated or unsaturated fatty acids having 16 to 24 carbon atoms, and in particular from the group of hexaglycerol di-fatty acid esters of stearic acid.

11. The cosmetic composition according to one of the preceding points, wherein the second polyglycerol di-fatty acid ester C is selected from the group of compounds with the INCI name PEG-3 Distearate.

12. The cosmetic composition according to one of the preceding points, wherein the second polyglycerol di-fatty acid ester C, based on the total weight of the compositions, is included in amounts of 0.2 to 8.0% by weight, preferably of 0.4 to 6.0% by weight, and in particular of 0.6 to 4.0% by weight.

13. The cosmetic composition according to one of the preceding points, wherein in the general formula (I) the groups R₁ and R₂ independently of one another stand for an unsaturated alkyl group having 16 to 26 carbon atoms, preferably for an unsaturated alkyl group having 18 to 24 carbon atoms.

14. The cosmetic composition according to one of the preceding points, wherein the fatty acid ester of the general formula (I), based on the total weight of the compositions, is included in amounts of 0.05 to 3.0% by weight, preferably in amounts of 0.1 to 2.0% by weight, and in particular in amounts of 0.2 to 1.5% by weight.

15. The cosmetic composition according to one of the preceding points, wherein the thickener is selected from the group of organic polymers.

16. The cosmetic composition according to one of the preceding points, wherein the composition includes as a thickener at least one copolymer of 2-methyl-2[(1-oxo-2-propenyl)amino]-1-propanesulfonic acid, preferably a copolymer of 2-methyl-2[(1-oxo-2-propenyl)amino]-1-propanesulfonic acid and vinylpyrrolidone.

17. The cosmetic composition according to one of the preceding points, wherein the composition includes as a thickener at least one homopolyacrylic acid or salts thereof.

18. The cosmetic composition according to one of the preceding points, wherein the composition includes as a thickener at least one cellulose derivative, preferably a cellulose derivative from the group comprising methyl cellulose, ethyl cellulose, hydroxyalkyl cellulose, methyl hydroxyalkyl celluloses, and carboxymethylcellulose.

19. The cosmetic composition according to one of the preceding points, wherein the composition includes xanthan as a thickener.

20. The cosmetic composition according to one of the preceding points, wherein the thickener is selected from the group of inorganic thickeners, in particular from the group of hydrophobized clay substances.

21. The cosmetic composition according to one of the preceding points, wherein the thickener, based on the total weight of the compositions, is included in amounts of 0.05 to 3.0% by weight, preferably of 0.1 to 2.0% by weight, and in particular of 0.2 to 1.5% by weight.

22. The cosmetic composition according to one of the preceding points, wherein the composition includes at least two, preferably at least four, and in particular at least eight cosmetically active substances.

23. The cosmetic composition according to one of the preceding points, wherein the cosmetically active substance is selected from the group of vitamins, provitamins, and vitamin precursors, plant extracts, organic UV filters, and skin-lightening active substances.

24. The cosmetic composition according to one of the preceding points, wherein the composition includes as the cosmetically active substance at least one organic UV filter from the group of substituted dibenzoylmethanes, diphenylacrylic acid esters, salicylic acid esters, and benzimidazoles.

25. The cosmetic composition according to one of the preceding points, wherein the composition includes as the cosmetically active substance at least one, preferably at least two, preferably at least three, and in particular at least four organic UV filters from the group comprising 3,3,5-trimethylcyclohexyl salicylate, 2-phenylbenzimidazole-5-sulfonic acid and salts thereof, 1-(4-tert-butylphenyl)-3-(4-methoxyphenyl)propane-1,3-dione, 1,1′-diphenylacrylonitrilic acid-2-ethylhexyl esters, and phenylene-1,4-bis(2-benzimidazyl)-3,3′-5,5′-tetrasulfonic acid and salts thereof.

26. The cosmetic composition according to one of the preceding points, wherein the composition includes as cosmetically active substance e) at least one organic UV filter and the organic UV filter, based on the total weight the compositions, is included in amounts of 1.0 to 35% by weight, preferably of 2.0 to 30% by weight, and in particular of 5.0 to 25% by weight.

27. The cosmetic composition according to one of the preceding points, wherein the composition includes as cosmetically active substance e) at least one skin-lightening active substance from the group comprising ascorbic acid, 2-(4-hydroxyphenoxy)propionic acid, phenylethyl resorcinol, octadecenedioic acid, Olea europaea extract, ascorbyl tetraisopalmitate, magnesium ascorbyl phosphate, and ascorbyl glucoside.

28. The cosmetic composition according to one of the preceding points, wherein cosmetically active substance e), based on the total weight of the composition, is included in amounts of 2.0 to 35% by weight, preferably of 4.0 to 30% by weight, and in particular of 6.0 to 25% by weight.

29. The cosmetic composition according to one of the preceding points, wherein the composition includes furthermore
g) a linear fatty alcohol, preferably a linear fatty alcohol having 14 to 18 carbon atoms, in particular a linear fatty alcohol having 16 carbon atoms.

30. The cosmetic composition according to point 29, wherein the linear fatty alcohol, based on the total weight of the compositions, is included in amounts of 0.01 to 5.0% by weight, preferably in amounts of 0.05 to 3.0% by weight, and in particular in amounts of 0.1 to 1.0% by weight.

31. The cosmetic composition according to one of the preceding points, wherein the composition includes furthermore
h) a linear alkane having 11 to 18 carbon atoms, preferably a linear alkane having 11 to 13 carbon atoms, in particular a linear alkane having 11 and/or 13 carbon atoms.

32. The cosmetic composition according to point 31, wherein the linear alkane, based on the total weight of the compositions, is included in amounts of 0.1 to 10% by weight, preferably of 0.2 to 7.0% by weight, and in particular of 0.5 to 5.0% by weight.

33. Use of a combination, comprising
a) at least one monoglycerol fatty acid ester A,
b) at least one first polyglycerol di-fatty acid ester B,
c) at least one second polyglycerol di-fatty acid ester C different from the first polyglycerol di-fatty acid ester,
d) at least one fatty acid ester of the general formula (I)

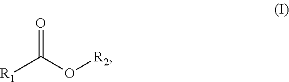

the R₁ and R₂ groups independently of one another stand for a saturated or unsaturated alkyl groups having 16 to 26 carbon atoms,
to improve the storage stability of a cosmetic composition including at least one

UV filter;
to improve the UV protective effect of a cosmetic composition including at least one UV filter;
to improve the absorption of a cosmetic composition including at least one UV filter;
to improve the spreadability and to reduce the tackiness of a cosmetic composition including at least one UV filter.

34. A method for cosmetic skin care in which a cosmetic composition according to one of points 1 to 32 is applied to the skin.

A first subject of the present invention is a cosmetic composition in the form of an oil-in-water emulsion, including:
a) at least one monoglycerol fatty acid ester A,
b) at least one first polyglycerol di-fatty acid ester B,
c) at least one second polyglycerol di-fatty acid ester C different from the first polyglycerol di-fatty acid ester B,
d) at least one fatty acid ester of the general formula (I)

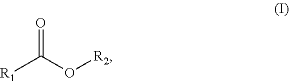

where the R₁ and R₂ groups independently of one another stand for a saturated or unsaturated alkyl groups having 16 to 26 carbon atoms,
e) at least one thickener,
f) at least one cosmetically active substance.

The cosmetic compositions of the invention are present as oil-in-water emulsion and therefore comprise an aqueous portion. The composition of the invention preferably has a viscous liquid form. Preferred cosmetic compositions are characterized, based on their total weight, by a water content of 30 to 82% by weight, preferably 35 to 75% by weight, in particular 45 to 60% by weight.

Apart from water, the compositions of the invention can include, based on their total weight, furthermore 1 to 15% by weight, preferably 2 to 12, and in particular 3 to 10% by weight of at least one alcohol. Preferred alcohols are glycol, 1,2-propylene glycol, 1,3-propylene glycol, 2-methyl-1,3-propanediol, glycerol, 1,2-butylene glycol, 1,3-butylene glycol, 1,4-butylene glycol, pentylene glycols, 1,2-hexanediol, 1,6-hexanediol, 1,2,6-hexanetriol, 1,2-octanediol, 1,8-octanediol, dipropylene glycol, tripropylene glycol, diglycerol, triglycerol, erythritol, sorbitol, polyethylene glycol, and mixtures of the aforementioned substances. Particularly preferably, the compositions of the invention include glycerol as the alcohol.

The cosmetic compositions include at least one monoglycerol fatty acid ester A as their first essential component.

C₁₆₋₂₂ carboxylic acids in particular, preferably saturated, linear C₁₆₋₂₂ carboxylic acids, are suitable as fatty acids. The monoglycerol fatty acid ester A is selected with particular preference from the group of mono-, di-, or triesters of glycerol with behenic acid. An exemplary monoglycerol fatty acid ester A, especially advantageous with respect to its action, is a mixture of mono-, di-, and triglycerides of behenic acid. Mixtures, comprising 10 to 25% by weight of monoglyceryl behenate, 40 to 65% by weight of diglyceryl behenate, and 25 to 50% by weight of trigylceryl behenate, are very especially preferred.

The monoglycerol fatty acid ester A is selected with particular preference from the group of compounds with the INCI name Glyceryl Behenate. A suitable ester is obtainable under the trade name Compritol® 888 CG (from Gattefosse).

The weight proportion of monoglycerol fatty acid ester A, in terms of the total weight of the cosmetic compositions, is preferably 0.01 to 6.0% by weight, preferably 0.1 to 4.0% by weight, and in particular of 0.5 to 2.0% by weight.

A further essential component of the cosmetic compositions is polyglycerol di-fatty acid ester B.

Exemplary polyglycerol di-fatty acid esters B are polyglyceryl-2 diisostearate, polyglyceryl-2 dioleate, polyglyceryl-2 dipolyhydroxystearate, polyglyceryl-2 distearate, polyglyceryl-3 dicaprate, polyglyceryl-3 dicocoate, polyglyceryl-3 dihydroxystearate, polyglyceryl-3 diisostearate, polyglyceryl-3 dioleate, and polyglyceryl-3 distearate.

Preferred polyglycerol di-fatty acid esters B are selected from the group of di-, tri-, or tetraglycerol di-fatty acid esters, preferably from the group of triglycerol di-fatty acid esters.

Based on their cosmetic and technical action, preferred are polyglycerol di-fatty acid esters B from the group of triglycerol di-fatty acid esters of saturated or unsaturated fatty acids having 16 to 30 carbon atoms, preferably from the group of triglycerol di-fatty acid esters of fatty acids from the group comprising palmitic acid, palmitoleic acid, oleic acid, cerotic acid, and melissic acid.

Exemplary polyglycerol di-fatty acid esters B are polyglyceryl-2 dipalmitate, polyglyceryl-2 dipalmitoleate, polyglyceryl-2 dioleate, polyglyceryl-2 dicerotinate, polyglyceryl-2 dimelissinate, polyglyceryl-3 dipalmitate, polyglyceryl-3 dipalmitoleate, polyglyceryl-3 dioleate, polyglyceryl-3 dicerotinate, polyglyceryl-3 dimelissinate, polyglyceryl-4 dipalmitate, polyglyceryl-4 dipalmitoleate, polyglyceryl-4 dioleate, polyglyceryl-4 dicerotinate, and polyglyceryl-4 dimelissinate. Polyglyceryl-3 dipalmitate, polyglyceryl-3 dicerotinate, and polyglyceryl-3 dimelissinate are particularly preferred.

Palmitic acid, palmitoleic acid, oleic acid, cerotic acid, and melissic acid are components of beeswax and the aforementioned polyglycerol di-fatty acid esters can be prepared, inter alia, by reacting polyglycerol with the free acids of beeswax. Particularly preferred polyglycerol di-fatty acid esters B are selected from the group of compounds with the INCI name PEG-3 Beeswax.

The weight proportion of polyglycerol di-fatty acid ester B, in terms of the total weight of the cosmetic compositions, is preferably 0.05 to 3.0% by weight, preferably 0.1 to 2.0% by weight, and in particular 0.2 to 1.5% by weight.

The cosmetic compositions include as their third essential component a second polyglycerol di-fatty acid ester C different from the first polyglycerol di-fatty acid ester B.

Exemplary polyglycerol di-fatty acid esters C include polyglyceryl-4 dilaurate, polyglyceryl-5 dilaurate, polyglyceryl-5 dioleate, polyglyceryl-6 dicaprate, polyglyceryl-6 diisostearate, polyglyceryl-6 dioleate, polyglyceryl-6 dipalmitate, polyglyceryl-6 distearate, polyglyceryl-10 didecanoate, polyglyceryl-10 diisostearate, polyglyceryl-10 dilaurate, polyglyceryl-10 dimyristate, polyglyceryl-10 dioleate, polyglyceryl-10 dipalmitate, and polyglyceryl-10 distearate.

Based on the advantageous cosmetic and technical action, the second polyglycerol di-fatty acid ester C is selected preferably from the group of penta-, hexa-, or heptaglycerol di-fatty acid esters, preferably from the group of hexagylcerol di-fatty acid esters.

Based on their cosmetic and technical action, preferred are polyglycerol di-fatty acid esters C from the group of hexaglycerol di-fatty acid esters of saturated or unsaturated fatty acids having 16 to 30 carbon atoms, preferably from the group of hexaglycerol di-fatty acid esters of saturated or unsaturated fatty acids having 16 to 24 carbon atoms, and preferably in particular from the group of hexaglycerol di-fatty acid esters of stearic acid.

Particularly preferred polyglycerol di-fatty acid esters C are selected from the group of compounds with the INCI name PEG-6 Distearate.

The weight proportion of polyglycerol di-fatty acid ester C, in terms of the total weight of the cosmetic compositions, is preferably 0.2 to 8.0% by weight, preferably 0.4 to 6.0% by weight, and in particular 0.6 to 4.0% by weight.

A further essential component of the cosmetic compositions is the fatty acid ester of the general formula (I). Based on their cosmetic and technical advantages, such fatty acid esters are preferred in which the R₁ and R₂ group in the general formula (I) independently of one another stand for an unsaturated alkyl group having 16 to 26 carbon atoms, preferably for an unsaturated alkyl group having 18 to 24 carbon atoms.

Particularly preferred fatty acid esters of the general formula (I) are selected from the group of compounds with the INCI name Jojoba Esters.

Preferably, the weight proportion of the fatty acid ester of the general formula (I), in terms of the total weight of the compositions, is 0.05 to 3.0% by weight, preferably 0.1 to 2.0% by weight, and in particular 0.2 to 1.5% by weight.

Based on the advantageous cosmetic action and the increased chemical and physical storage stability, it is particularly preferable to use mixtures of polyglycerol di-fatty acid esters B, polyglycerol di-fatty acid esters C, and fatty acid esters of the general formula (I), comprising, based on the total weight of the mixture,
5.0 to 10% by weight of polyglycerol di-fatty acid ester B,
50 to 75% by weight of polyglycerol di-fatty acid ester C, and
10 to 25% by weight of the fatty acid ester of the general formula (I).

In summary, cosmetic compositions in the form of an oil-in-water emulsion are preferred that include:
a) 0.01 to 6.0% by weight of at least one monoglycerol fatty acid ester A from the group of mono-, di-, or triesters of glycerol with behenic acid,
b) 0.05 to 3.0% by weight of at least one first polyglycerol di-fatty acid ester B from the group of di-, tri-, or tetraglycerol di-fatty acid esters of saturated or unsaturated fatty acids having 16 to 30 carbon atoms,
c) 0.2 to 8.0% by weight of at least one second polyglycerol di-fatty acid ester C, different from the first polyglycerol di-fatty acid ester, from the group of penta-, hexa-, or heptaglycerol di-fatty acid esters of saturated or unsaturated fatty acids having 16 to 30 carbon atoms,
d) 0.05 to 3.0% by weight of at least one fatty acid ester of the general formula (I)

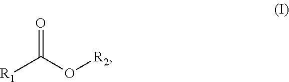

where the R₁ and R₂ groups independently of one another stand for an unsaturated alkyl groups having 16 to 26 carbon atoms, and
e) at least one thickener, and
f) at least one cosmetically active substance.

In a preferred embodiment, the cosmetic compositions include as an optional component furthermore a linear fatty alcohol, preferably a linear fatty alcohol having 14 to 18 carbon atoms, in particular a linear fatty alcohol having 16 carbon atoms.

Based on the total weight of the compositions, the weight proportion of the linear fatty alcohol is preferably 0.01 to 5.0% by weight, preferably 0.05 to 3.0% by weight, and in particular 0.1 to 1.0% by weight.

To establish a suitable viscosity and to improve further the storage stability, the cosmetic compositions include at least one thickener as their fifth essential component. The thickener is included in the cosmetic compositions, based on the total weight, preferably in amounts of 0.05 to 3.0% by weight, preferably of 0.1 to 2.0% by weight, and in particular of 0.2 to 1.5% by weight.

The thickener can be selected, for example, from the polymeric thickeners known under the following INCI names: Acrylamides Copolymer, Acrylamide/Sodium Acrylate Copolymer, Acrylamide/Sodium Acryloyldimethyltaurate Copolymer, Acrylates/Acetoacetoxyethyl Methacrylate Copolymer, Acrylates/Beheneth-25 Methacrylate Copolymer, Acrylates/C10-30 Alkyl Acrylate Crosspolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-25 Acrylate Copolymer, Acrylates/Palmeth-25 Itaconate Copolymer, Acrylates/Steareth-50 Acrylate Copolymer, Acrylates/Steareth-20 Itaconate Copolymer, Acrylates/Steareth-20 Methacrylate Copolymer, Acrylates/Stearyl Methacrylate Copolymer, Acrylates/Vinyl Isodecanoate Crosspolymer, Acrylic Acid/Acrylonitrogens Copolymer, Agar, Agarose, Alcaligenes Polysaccharides, Algin, Alginic Acid, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Polyacryloyldimethyl Taurate, Amylopectin, Ascorbyl Methylsilanol Pectinate, Astragalus Gummifer Gum, Attapulgite, Avena Sativa (Oat) Kernel Flour, Bentonite, Butoxy Chitosan, Caesalpinia Spinosa Gum, Calcium Alginate, Calcium Carboxymethyl Cellulose, Calcium Carrageenan, Calcium Potassium Carbomer, Calcium Starch Octenylsuccinate, C20-40 Alkyl Stearate, Carbomer, Carboxybutyl Chitosan, Carboxymethyl Chitin, Carboxymethyl Chitosan, Carboxymethyl Dextran, Carboxymethyl Hydroxyethylcellulose, Carboxymethyl Hydroxypropyl Guar, Cellulose Acetate Propionate Carboxylate, Cellulose Gum, Ceratonia Siliqua Gum, Cetyl Hydroxyethylcellulose, Cholesterol/HDI/Pullulan Copolymer, Cholesteryl Hexyl Dicarbamate Pullulan, Cyamopsis Tetragonoloba (Guar) Gum, Diglycol/CHDM/Isophthalates/SIP Copolymer, Dihydrogenated Tallow Benzylmonium Hectorite, Dimethicone Crosspolymer-2, Dimethicone Propyl PG-Betaine, DMAPA Acrylates/Acrylic Acid/Acrylo nitro gens Copolymer, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hydrated Silica, Hydrogenated Potato Starch, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulo se Stearoxy Ether, Hydroxystearamide MEA, Isobutylene/Sodium Maleate Copolymer, Lithium Magnesium Silicate, Lithium Magnesium Sodium Silicate, Macrocystis Pyrifera (Kelp), Magnesium Alginate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Montmorillonite, Moroccan Lava Clay, Natto Gum, Nonoxynyl Hydroxyethylcellulose, Octadecene/MA Copolymer, Pectin, PEG-800, PEG-Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TM MG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 Methyl Glucose Trioleate, PEG-180/0 ctoxynol-40/TMMG Copolymer, PEG-150 Pentaerythrityl Tetrastearate, PEG-4 Rapeseedamide, PEG-150/Stearyl Alcohol/SMDI Copolymer, Polyacrylate-3, Polyacrylic Acid, Polycyclopentadiene, Polyether-1, Polyethylene/Isopropyl Maleate/MA Copolyol, Polymethacrylic Acid, Polyquaternium-52, Polyvinyl Alcohol, Potassium Alginate, Potassium Aluminum Polyacrylate, Potassium Carbomer, Potassium Carrageenan, Potassium Polyacrylate, Potato Starch Modified, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Rhizobian Gum, Ricinoleic Acid/Adipic Acid/AEEA Copolymer, Sclerotium Gum, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Cyclodextrin Sulfate, Sodium Hydroxypropyl Starch Phosphate, Sodium Isooctylene/MA Copolymer, Sodium Magnesium Fluorosilicate, Sodium Polyacrylate, Sodium Polyacrylate Starch, Sodium Polyacryloyldimethyl Taurate, Sodium Polymethacrylate, Sodium Polystyrene Sulfonate, Sodium Silicoaluminate, Sodium Starch Octenylsuccinate, Sodium Stearoxy PG-Hydroxyethylcellulose Sulfonate, Sodium Styrene/Acrylates Copolymer, Sodium Tauride Acrylates/Acrylic Acid/Acrylonitrogens Copolymer, Solanum Tuberosum (Potato) Starch, Starch/Acrylates/Acrylamide Copolymer, Starch Hydroxypropyltrimonium Chloride, Steareth-60 Cetyl Ether, Steareth-100/PEG-136/HDI Copolymer, Sterculia Urens Gum, Synthetic Fluorphlogopite, Tamarindus Indica Seed Gum, Tapioca Starch, TEA-Alginate, TEA-Carbomer, Triticum Vulgare (Wheat) Starch, Tromethamine Acrylates/Acrylonitrogens Copolymer, Tromethamine Magnesium Aluminum Silicate, Welan Gum, Yeast Beta-Glucan, Yeast Polysaccharides, Zea Mays (Corn) Starch.

The polymeric thickener is selected particularly preferably from among polymeric, anionic, amphiphilic thickeners, particularly preferably from those with the INCI names: Acryl ate s/B eheneth-25 Methacrylate Copolymer, Acrylates/C10-30 Alkyl Acrylate Crosspolymer, Acrylates/Ceteth-20 Itaconate Copolymer, Acrylates/Ceteth-20 Methacrylate Copolymer, Acryl ate s/Laureth-25 Methacrylate Copolymer, Acrylates/Palmeth-20 Acrylate Copolymer, Acrylates/Palmeth-25 Acrylate Copolymer, Acrylates/Palmeth-25 Itaconate Copolymer, Acrylates/Steareth-50 Acrylate Copolymer, Acrylates/Steareth-20 Itaconate Copolymer, Acrylates/Steareth-20 Methacrylate Copolymer, Acrylates/Stearyl Methacrylate Copolymer, Acrylates/Vinyl Isodecanoate Crosspolymer, and Acrylates/Steareth-20 Methacrylate Crosspolymer.

In a first preferred embodiment, the thickener is selected from the group of organic polymers.

A first preferred organic thickener is the copolymers of 2-methyl-2[(1-oxo-2-propenyl)amino]-1-propanesulfonic acid, copolymers of 2-methyl-2[(1-oxo-2-propenyl)amino]-1-propanesulfonic acid and vinylpyrrolidone being particularly preferred. These copolymers are preferably crosslinked.

Crosslinked copolymers of vinylpyrrolidone and 2-methyl-2[(1-oxo-2-propenyl)amino]-1-propanesulfonic acid are obtainable, for example, by copolymerization of the two monomers in the presence of ammonium hydroxide and a crosslinking agent. The crosslinking may occur with the aid of polyolefinically unsaturated compounds, for example, divinylbenzene, tetraallyloxyethane, methylene bisacrylamide, diallyl ether, polyallyl polyglyceryl ether, or allyl ethers of sugars, or sugar derivatives such as erythritol, pentaerythritol, arabitol, mannitol, sorbitol, sucrose, or glucose. Methylene bisacrylamide is a preferred crosslinking agent.

An especially suitable copolymer is the commercial product Aristoflex® AVC (from Clariant), which is present as a white powder in the form of the ammonium salt.

The compositions of the invention include the copolymer of 2-methyl-2[(1-oxo-2-propenyl)amino]-1-propanesulfonic acid preferably in amounts of 0.05 to 3.0% by weight, preferably of 0.1 to 2.0% by weight, and in particular of 0.2 to 1.5 by weight.

The homopolyacrylic acid or salts thereof form a group of further preferred organic thickeners.

Preferred in particular are the homopolyacrylic acids (INCI: Carbomer), which are obtainable commercially under the name Carbopol® (from Lubrizol) as various types.

Suitable, furthermore, are the crosslinked homopolyacrylic acids that can be crosslinked, for example, by pentaerythritol or sucrose allyl ether or by propylene. Suitable crosslinked homopolyacrylic acids (INCI: Carbomer) are obtainable commercially under the name Carbopol Ultrez®, in particular Carbopol Ultrez® 30 (from Lubrizol).

The compositions of the invention preferably include the homoacrylic acid in amounts of 0.05 to 3.0% by weight, preferably of 0.1 to 2.0% by weight, and in particular of 0.2 to 1.0% by weight.

Celluloses and cellulose derivatives, in particular cellulose derivatives, form a third group of preferred organic thickeners. This specific group of thickeners includes, for example, calcium carboxymethyl cellulose, carboxymethyl hydroxyethylcellulose, carboxymethyl hydroxypropyl guar, cellulose acetate propionate carboxylate, cellulose gum, cetyl hydroxyethylcellulose, hydroxybutyl methylcellulose, hydroxyethylcellulose, hydroxyethyl ethylcellulose, hydroxypropyl cellulose, hydroxypropyl methylcellulose, hydroxypropyl methyl cellulose stearoxy ether, methyl cellulose, methyl ethylcellulose, methyl hydroxyethyl cellulose, microcrystalline cellulose, nonoxynyl hydroxyethyl cellulose, sodium cellulose sulfate, and sodium stearoxy PG-hydroxyethyl cellulose. Particularly preferred is the use of cellulose derivatives from the group comprising methyl cellulose, ethyl cellulose, hydroxyalkyl cellulose, methyl hydroxyalkyl celluloses, and carboxymethylcellulose.

The compositions of the invention include the celluloses and cellulose derivatives, in particular the cellulose derivatives, preferably in amounts of 0.05 to 2.0% by weight, preferably of 0.1 to 1.5% by weight, and in particular of 0.2 to 1.0% by weight.

In a further preferred embodiment, the cosmetic compositions include xanthan, preferably xanthan with the INCI name Xanthan Gum, as the organic thickener.

The compositions of the invention include xanthan preferably in amounts of 0.05 to 2.0% by weight, preferably of 0.1 to 1.5% by weight, and in particular of 0.2 to 1.0% by weight.

Alternatively or in addition to the previously described organic thickeners, the cosmetic compositions can include inorganic thickeners, in particular inorganic thickeners from the group of the hydrophobized clay substances.

The cosmetic compositions include the inorganic thickener in amounts of 0.05 to 2.0% by weight, preferably of 0.1 to 1.5% by weight, and in particular of 0.2 to 1.0% by weight.

Inorganic thickeners preferred according to the invention are selected from hydrophobized clay minerals and silicic acid (in particular pyrogenic silicic acid).

Preferred hydrophobized clay minerals are selected from hydrophobized montmorillonites, hydrophobized hectorites, and hydrophobized bentonites, particularly preferably from Disteardimonium Hectorite, Stearalkonium Hectorite, Quaternium-18 Hectorite, and Quaternium-18 Bentonite. The commercial thickeners provide these hydrophobized clay minerals in the form of a gel in an oil component, preferably in cyclomethicone and/or a non-silicone oil component such as, e.g., propylene carbonate. Gels of this type are obtainable, for example, under the trade name Benton® or Thixogel®.

The cosmetic compositions include at least one cosmetically active substance as a further essential component. The term ‘cosmetically active substance’ in this case describes individual cosmetically active substances as well as substance mixtures that cannot be separated into their individual components and, in the production of cosmetic compositions, are actually regarded and used as individual components. An example of a substance mixture of this type is cosmetically active plant extracts.

The weight proportion of the cosmetically active substances, in terms of the total weight of the compositions, is preferably 2.0 to 35% by weight, preferably 4.0 to 30% by weight, and in particular 6.0 to 25% by weight.

Examples of preferred cosmetic active substances, in addition to the aforementioned plant extracts, are in particular vitamins, provitamins, and vitamin precursors, organic UV filters, and skin-lightening active substances.

The plant extracts form a first group of preferred cosmetically active substances. Otherwise typical preservatives can be avoided optionally by the use of plant extracts as cosmetically active substances d). Preferred above all according to the invention are the extracts from green tea, oak bark, nettle, hamamelis, hops, henna, chamomile, burdock root, horsetail, hawthorn, linden blossom, almond, aloe vera, pine needles, horse chestnut, sandalwood, juniper, coconut, mango, apricot, lemon, wheat, kiwi, melon, orange, grapefruit, salvia, rosemary, birch, mallow, valerian, cuckoo flower, wild thyme, yarrow, thyme, melissa, restharrow, coltsfoot, marshmallow, meristem, ginseng, coffee, cocoa, moringa, ginger root, and ayurvedic plant extracts such as, for example, Aegle marmelos (bilwa), Cyperus rotundus (nagar motha), Emblica officinalis (amalaki), Morinda citrifolla (ashyuka), Tinospora cordifolia (guduchi), Santalum album (chandana), Crocus sativus (kumkuma), Cinnamonum zeylanicum and Nelumbo nucifera (kamala), sweet grasses such as wheat, barley, rye, oats, spelt, corn, various varieties of sorghum (proso millet, crabgrass, foxtail millet as examples), sugar cane, rye grass, meadow foxtail, oatgrass, bentgrass, meadow fescue, moor grass, bamboo, cotton grass, fountain grasses, Andropogonodeae (Imperata cylindrica, also known as flame grass or cogon grass), buffalo grass, cord grasses, dog's tooth grasses, love grasses, Cymbopogon (lemongrass), Oryzeae (rice), Zizania (wild rice), beach grass, blue oat grasses, soft grasses, quaking grasses, meadow grasses, couch grass, and Echinacea, in particular Echinacea purpurea (L.) Moench, and all types of wine and pericarp of Litchi chinensis.

Extracts from Quassia amara, Opuntia ficus indica, Bambusa vulgaris, and Pisum sativum are very particularly preferred.

Quassia amara (Brazilian quassia tree, quassia tree, bitterwood) is a plant species in the family Simaroubaceae.

Opuntia ficus indica is a plant species of the genus Opuntia from the family of cactus plants (Cactaceae), which is native to India.

Bambusa vulgaris (gold bamboo) is a plant species from the family of sweet grasses (Poaceae), which is occurs predominantly in Southeast Asia and Africa.

Pisum sativum is a plant species of the pea genus (Pisum) from the legume family (Fabaceae) and stems originally from Asia Minor.

Bambusa vulgaris extract and/or Pisum sativum extract support collagen and elastin synthesis in the skin and contribute considerably, in particular in combination with an extract from Opuntia ficus indica, to achieving an especially uniform and elastic appearance of the skin. Cosmetic compositions including a combination of all three of these particularly preferred plant extracts are therefore particularly preferred.

Vitamins, provitamins, and vitamin precursors form a further preferred group of cosmetically active substances. Preferred cosmetic compositions are characterized in that the cosmetically active substance comprises a vitamin, provitamin, or a compound designated as a vitamin precursor from the vitamin groups A, B, C, E, H, and K and the esters of the aforementioned substances.

The group of substances designated as vitamin A include retinol and 3,4-didehydroretinol. Beta-carotene is the retinol provitamin. Suitable vitamin A components according to the invention are, for example, vitamin A acid and esters thereof, vitamin A aldehyde, and vitamin A alcohol and esters thereof such as the palmitate and acetate.

The vitamin B group or the vitamin B complex includes, inter alia: Vitamin B1 (thiamine), vitamin B2 (riboflavin), and vitamin B3 (the compounds nicotinic acid and nicotinic acid amide (niacinamide) are often included under this term), and vitamin B5 (pantothenic acid, panthenol, and pantolactone).

Panthenol and/or pantolactone are used with preference within the scope of the group. Panthenol derivatives usable according to the invention are particularly the esters and ethers of panthenol and cationically derivatized panthenols. Individual representatives are, for example, panthenol triacetate, panthenol monoethyl ether, and the monoacetate thereof, as well as cationic panthenol derivatives. Pantothenic acid is preferably used in the present invention as a derivative in the form of the more stable calcium salts and sodium salts (Ca pantothenate, Na pantothenate).

Vitamin B6 (pyridoxine as well as pyridoxamine and pyridoxal).

Vitamin C (ascorbic acid) is used preferentially in the agents of the invention. The use in the form of the palmitic acid ester, glucosides, or phosphates can be preferred. The use in combination with tocopherols can likewise be preferred. The use of ascorbyl tetraisopalmitate and magnesium ascorbyl phosphate is particularly preferred.

Vitamin E (tocopherols, especially alpha-tocopherol). Particularly preferred agents of the invention are tocopherol and its derivatives, which include in particular the esters such as acetate, nicotinate, phosphate, and succinate.

Vitamin F. The term “vitamin F” is usually understood to mean essential fatty acids, in particular linoleic acid, linolenic acid, and arachidonic acid.

Vitamin H. Vitamin H is the name for the compound (3aS,4S,6aR)-2-oxohexahydrothienol[3,4-d]-imidazole-4-valeric acid, although the trivial name biotin has become accepted in the meantime.

The compositions of the invention preferably include vitamins, provitamins, and vitamin precursors from the groups A, B, E, and/or H. Panthenol, pantolactone, pyridoxine, and their derivatives, as well as nicotinic acid amide and biotin are likewise preferred.

UV filter substances form the third group of preferred cosmetically active substances.

Both the agents themselves and treated fibers can be protected from the harmful effects of UV radiation by addition of a UV filter. Preferably, therefore, at least one UV filter is added to the agent. Suitable UV filters are not subject to any general restrictions with respect to their structure and their physical properties. Rather, all UV filters that can be used in the cosmetics field and whose absorption maximum is in the UVA (315-400 nm), in the UVB (280-315 nm), or in the UVC (<280 nm) range are suitable. UV filters with an absorption maximum in the UVB range, in particular in the range of about 280 to about 300 nm, are particularly preferred.

Examples of UV filters usable according to the invention are 4-aminobenzoic acid, N,N,N-trimethyl-4-(2-oxoborn-3-ylidenemethyl)ani line methyl sulfate, 3,3,5-trimethyl cyclo hexyl salicylate (homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and the potassium, sodium, and triethanolamine salts thereof (Phenylbenzimidazole Sulfonic Acid; Parsol® HS; Neo Heliopan® Hydro), 3,3′-(1, 4-phenylenedimethylene)-bi s(7,7-dimethyl-2-oxo-bicyclo [2.2.1]hept-1-yl-methanesulfonic acid) and salts thereof, 1-(4-tert-butylphenyl)-3-(4-methoxyphenyl)-propane-1,3-dione (Butyl Methoxydibenzoylmethane; Parsol® 1789, Eusolex® 9020), α-(2-oxoborn-3-ylidene)toluene-4-sulfonic acid and salts thereof, ethoxylated 4-aminobenzoic acid ethyl ester (PEG-25 PABA; Uvinul® P 25), 4-dimethylaminobenzoic acid-2-ethylhexyl ester (Octyl Dimethyl PABA; Uvasorb® DMO, Escalol® 507, Eusolex® 6007), salicylic acid-2-ethylhexyl ester (Octyl Salicylate; Escalol® 587, Neo Heliopan® OS, Uvinul® 018), 4-methoxycinnamic acid isopentyl ester (Isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone-5-sulfonic acid and the sodium salt thereof (Benzophenone-4; Uvinul® MS 40; Uvasorb® S 5), 3-(4′-methylbenzylidene)-D,L-camphor (4-Methylbenzylidene Camphor; Parsol® 5000, Eusolex® 6300), 3-benzylidene camphor (3-Benzylidene Camphor), 4-isopropylbenzyl salicylate, 2,4,6-trianilino-(p-carbo-2′-ethylhexyl-1′-oxi)-1,3,5-triazine, 3-imidazol-4-yl-acrylic acid and the ethyl esters thereof, polymers of the N-{(2 and 4)[2-oxoborn-3-ylidenemethyl]benzene}-acrylamide, 2,4-dihydroxybenzophenone (Benzophenone-1; Uvasorb® 20 H, Uvinul® 400), 1,1′-diphenylacrylonitrilic acid-2-ethyl hexyl esters (Octocrylene; Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodium sulfonate, 2-cyano-3,3-diphenylacrylic acid-2′-ethylhexyl ester, and phenylene-1,4-bis(2-benzimidazyl)-3,3′-5,5′-tetrasulfonic acid-bis-sodium salt (Disodium Phenyl Dibenzimidazole Tetrasulfonate, Neo Heliopan AP).

The UV filters preferred according to the invention are substituted dibenzoylmethane, diphenylacrylic acid esters, salicylic acid esters, and benzimidazoles.

Particularly preferred organic UV filters are
3,3,5-trimethylcyclohexyl salicylate,
2-phenylbenzimidazole-5-sulfonic acid and salts thereof,
1-(4-tert-butylphenyl)-3-(4-methoxyphenyl)propane-1,3-dione,
1,1′-diphenylacrylonitrilic acid-2-ethylhexyl ester, preferably in combination with ethylenediaminetetraacetic acid and the salts thereof,
phenylene-1,4-bis(2-benzimidazyl)-3,3′-5,5′-tetrasulfonic acid and the salts thereof, preferably in combination with an amino acid, preferably in combination with arginine, histidine, ornithine, or lysine, in particular in combination with arginine.

Of course, combinations of organic UV filters can also be used in the cosmetic compositions of the invention. Preferred combinations of UV filters in this case refer in particular to the UV filters described heretofore as particularly preferred. Because of their excellent physical stability, the cosmetic compositions of the invention are especially suitable for producing a plurality of organic UV filters.

It is particularly preferred if the organic UV filter includes at least one, preferably at least two, preferably at least three, and in particular at least four organic UV filters from the group comprising 3,3,5-trimethylcyclohexyl salicylate, 2-phenylbenzimidazole-5-sulfonic acid and salts thereof, 1-(4-tert-butylphenyl)-3-(4-methoxyphenyl)propane-1,3-dione, 1,1′-diphenylacrylonitrilic acid-2-ethylhexyl esters, and phenyl-1,4-bi s(2-benzimidazyl)-3,3′-5,5′-tetrasulfonic acid and salts thereof

The weight proportion of the organic UV filters, in terms of the total weight of the compositions, is preferably 1.0 to 35% by weight, preferably 2.0 to 30% by weight, and in particular 5.0 to 25% by weight.

Skin-lightening active substances form a fourth group of particularly preferred cosmetic active substances. These active substances reduce or eliminate dark pigment spots on the skin. Preferred are amounts of 0.02 to 9.0% by weight, preferably 0.03 to 8.0% by weight, particularly preferably 0.04 to 7.0% by weight, and in particular 0.05 to 6.0% by weight of skin-lightening active substance e).

Particularly suitable skin-lightening active substances are selected from: ascorbic acid, ascorbic acid salts, ascorbic acid glycosides, ascorbic acid glycoside salts, organic ascorbic acid esters, salts of organic ascorbic acid esters, inorganic ascorbic acid esters, salts of organic ascorbic acid esters, 2-(4-hydroxyphenoxy) propionic acid, salts of 2-(4-hydroxyphenoxy) propionic acid, resorcinol derivatives, saturated or unsaturated, linear C₉₋₁₈ dicarboxylic acid (salts), extracts of Olea europaea, as well as mixtures of said active substances.

In a preferred embodiment, the compositions of the invention include as at least one skin-lightening active substance, therefore at least one active substance from the aforementioned group.

Alkali, ammonium, and alkaline earth salts are to be understood preferably as suitable ascorbic acid salts. Particularly preferred salts of ascorbic acid that can be used as a skin-lightening active substance of the invention are sodium hydroascorbate, sodium ascorbate, potassium hydroascorbate, potassium ascorbate, ammonium ascorbate, ammonium hydroascorbate, calcium ascorbate, calcium hydroascorbate, magnesium ascorbate, and magnesium hydroascorbate.

Suitable ascorbic acid esters within the context of the present invention can be selected preferably from esters of ascorbic acid with organic acids according to the following formula (I)

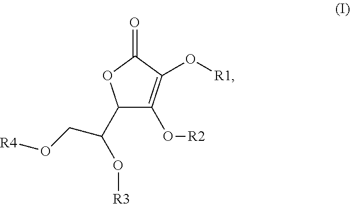

where one to four of the R1 to R4 groups stand for the group —C(O)—R and the other groups optionally stand for hydrogen; and R stands for a straight-chain or branched, saturated or unsaturated alk(en)yl group having 8 to 24, preferably having 10 to 20, and in particular having 13 to 17 carbon atoms.

Very particularly preferred esters of ascorbic acid with organic acids are the following compounds: ascorbyl palmitate, ascorbyl isopalmitate, ascorbyl tetraisopalmitate, ascorbyl stearate, ascorbyl isostearate, ascorbyl oleate, and/or ascorbyl linoleate. Because of its excellent skin penetration capability, ascorbyl tetraisopalmitate is a particularly preferred organic ascorbic acid ester.

Furthermore, suitable ascorbic acid esters within the context of the present invention are esters of ascorbic acid with inorganic acids, preferably with phosphoric acid or sulfuric acid. Particularly preferred according to the invention are ascorbyl phosphate and/or ascorbyl sulfate or the alkali or alkaline earth salts of ascorbyl phosphate and/or ascorbyl sulfate such as, for example, the compounds known under the INCI names Sodium Ascorbyl Phosphate, Magnesium Ascorbyl Phosphate, Sodium Ascorbyl Sulfate, and/or Magnesium Ascorbyl Sulfate. Magnesium Ascorbyl Phosphate is a particular preferred inorganic ascorbic acid ester because of its excellent stability in aqueous media.

Ascorbic acid derivatives with glycosidically linked sugars can also be used with particular preference according to the invention. Ascorbyl glucoside and/or one of its physiologically acceptable salts in particular have proven successful here.

Particularly preferred are compositions that include combinations of fat- and water-soluble ascorbic acid (derivatives) to increase the stability and effectiveness of the compositions. Particularly preferred are combinations of ascorbic acid with the aforementioned ascorbyl phosphate salts and/or ascorbyl glucoside and/or ascorbyl tetraisopalmitate.

A further preferred embodiment of the invention therefore is characterized in that the cosmetic compositions include as the skin-lightening active substance ascorbic acid, ascorbic acid salts, ascorbic acid glycosides, ascorbic acid glycoside salts, organic ascorbic acid esters, salts of organic ascorbic acid esters, inorganic ascorbic acid esters, and/or salts of organic ascorbic acid esters. Particularly preferred within this embodiment are compositions that include ascorbic acid and/or at least one compound known under the INCI names Ascorbyl Tetraisopalmitate, Magnesium Ascorbyl Phosphate, and/or Ascorbyl Glucoside.

Ascorbic acid and/or its aforementioned preferred derivatives and/or salts can be used in the cosmetic compositions of the invention preferably in amounts of 0.01 to 5.0% by weight, more preferably of 0.02 to 4.50% by weight, particularly preferably of 0.03 to 4.0% by weight, very particularly preferably of 0.04 to 3.50% by weight, and in particular of 0.05 to 3.0% by weight, the quantitative data referring to the total weight of the compositions.

Particularly preferred are compositions that include ascorbic acid and/or at least one compound known under the INCI names Ascorbyl Tetraisopalmitate, Magnesium Ascorbyl Phosphate, and/or Ascorbyl Glucoside in the aforementioned amounts. Preferred in particular are mixtures of ascorbic acid and ascorbyl glucoside.

A further preferred skin-lightening active substance is an extract of Olea europaea, in particular an extract from Olea europaea leaves as can be obtained commercially from various suppliers, for example, under the INCI name Olea europaea Leaf Extract.

The extract is produced by methods known per se from the state of the art preferably from olive tree leaves with a mixture of water and alcohol and subsequent hydrolysis of the extract.

Olive leaves include per gram of dry weight on average 60-90 mg of the polyphenol, oleuropein (in addition to phenols, flavonoids, organic acids, and hydrocarbons), which exhibits very high antioxidative activity. In addition, however, oleuropein also significantly reduces the cellular waste products lipofuscin, which are responsible for the formation of age spots.

In a further preferred embodiment, cosmetic compositions are characterized in that they include as a skin-lightening active substance at least one plant extract known under the INCI name Olea europaea Leaf Extract.

The extract of Olea europaea, in particular an extract known under the INCI name Olea europaea Leaf Extract, can be used in the cosmetic compositions preferably in an amount of 0.01 to 3.0% by weight, more preferably of 0.02 to 2.50% by weight, particularly preferably of 0.03 to 2.0% by weight, very particularly preferably of 0.04 to 1.50% by weight, and in particular of 0.05 to 1.0% by weight, the quantitative data referring to the total weight of the compositions.

In a further preferred embodiment, it can be of advantage to use mixtures of ascorbic acid and/or salts and/or derivatives of ascorbic acid with an extract of Olea europaea in the compositions of the invention.

Particularly preferred within this embodiment are mixtures of ascorbic acid, an ascorbic acid derivative known under the INCI name Ascorbyl Glucoside, and a plant extract known under the INCI name Olea europaea Leaf Extract. Such mixtures are commercially available, for example, under the name Illumiscin® from the company Rahn. Illumiscin® can be used in the compositions of the invention preferably as skin-lightening active substance e).

If the commercial product Illumiscin® is used as the skin-lightening active substance in the compositions of the invention, then its amount used is 0.10 to 8.0% by weight, more preferably 0.25 to 7.0% by weight, particularly preferably 0.50 to 6.0% by weight, very particularly preferably 0.75 to 5.0% by weight, and in particular 1.0 to 4.0% by weight, based in each case on the total weight of the compositions.

A further preferred skin-lightening active substance is selected from 2-(4-hydroxyphenoxy)propionic acid and/or salts of 2-(4-hydroxyphenoxy)propionic acid. 2-(4-Hydroxyphenoxy)propionic acid and salts thereof are supplied commercially in racemic form and in the form of their (R)- and (S)-enantiomers. 2-(4-Hydroxyphenoxy)propionic acid and/or salts of 2-(4-hydroxyphenoxy)propionic acid can be used in the compositions of the invention, preferably in terms of their total weight, preferably in amounts of 0.10 to 3.0% by weight, more preferably of 0.20 to 2.50% by weight, particularly preferably of 0.30 to 2.0% by weight, very particularly preferably of 0.40 to 1.50% by weight, and in particular of 0.50 to 1.25% by weight.

Understood according to the invention under 2-(4-hydroxyphenoxy)propionic acid are both the racemic mixture and (S)-2-(4-hydroxyphenoxy)propionic acid or (R)-2-(4-hydroxyphenoxy)propionic acid.

(R)-2-(4-Hydroxyphenoxy)propionic acid or a physiologically acceptable salt such as alkali or ammonium salts of (R)-2-(4-hydroxyphenoxy)propionic acid is preferred with respect to the excellent effectiveness against pigment spots of any type (age spots and/or freckles).

Suitable products are commercially available, for example, under the trade name Radianskin® PW LS 9918 from the company BASF.

It has been found that the treatment of highly pigmented skin with the compositions of the invention produced especially good results with respect to the lightening of pigment spots, if ascorbic acid and/or derivatives and/or salts thereof were combined with olive leaf extract and (R)-2-(4-hydroxyphenoxy)propionic acid. In particular, the content of lipofuscin in the skin is greatly reduced thereby and therefore age spots as well are lightened better or removed.

Particularly suitable in this regard was the combination of the commercial products Illumiscin® and Radianskin® PW LS 9918.

In a further preferred embodiment, cosmetic compositions are therefore characterized in that they include
ascorbic acid and ascorbyl glucoside,
at least one plant extract known under the INCI name Olea europaea Leaf Extract, and
(R)-2-(4-hydroxyphenoxy)propionic acid and/or the alkali metal or ammonium salts thereof.

Further resorcinol derivatives, in particular compounds known under the INCI name Phenylethyl Resorcinol, and/or saturated or unsaturated, linear C₉₋₁₈ dicarboxylic acid (salts), can be used as skin-lightening active substances within the meaning of the present invention; these can be used in each case in the compositions of the invention preferably in amounts of 0.01 to 3.0% by weight, more preferably of 0.05 to 2.50% by weight, particularly preferably of 0.10 to 2.0% by weight, very particularly preferably of 0.20 to 1.50% by weight, and in particular of 0.30 to 1.0% by weight, the quantitative data referring to the total weight of the compositions.

Particularly suitable saturated or unsaturated, linear C₉₋₁₈ dicarboxylic acid (salts) can be selected preferably from: 8-hexadecene-1,16-dicarboxylic acid, 7-tetradecene-1,14-dicarboxylic acid, 9-octadecene-1,18-dicarboxylic acid, 6-dodecene-1,12-dicarboxylic acid, 5-decene-1,10-dicarboxylic acid, decane diacid (sebacic acid), and/or nonane diacid (azelaic acid).

8-Hexadecene-1,16-dicarboxylic acid (INCI name Octadecenedioic Acid) is a metabolic product of yeast cells from selected mutant strains of the Candida genus; a fatty acid of pure plant origin is used as the starting substance, which is converted to the hydroxy fatty acid, which is then oxidized via the stage of fatty acid aldehyde to dicarboxylic acid.

The commercial product has a purity of 95%; the 8-hexadecene-1,16-dicarboxylic acid is present therein as a mixture of the cis- and trans-isomers and the cis-isomer predominates quantitatively. The product can include up to 3% by weight of oleic acid.

Very particularly preferred compositions include, based on their total weight, 0.05 to 3.0% by weight, preferably 0.07 to 2.0% by weight, more preferably 0.08 to 1.50% by weight, and in particular 0.10 to 1.0% by weight of 8-hexadecene-1,16-dicarboxylic acid (INCI name Octadecenedioic Acid).

In a further preferred embodiment, cosmetic compositions are characterized in that they include as the skin-lightening active substance at least one compound known under the INCI name Phenylethyl Resorcinol and/or at least one linear C₉₋₁₈ dicarboxylic acid, preferably 8-hexadecene-1,16-dicarboxylic acid, 7-tetradecene-1,14-dicarboxylic acid, 9-octadecene-1,18-dicarboxylic acid, 6-dodecene-1,12-dicarboxylic acid, 5-decene-1,10-dicarboxylic acid, decane diacid (sebacic acid), and/or nonane diacid (azelaic acid), very particularly preferably phenylethyl resorcinol and/or 9-octadecene-1,18-dicarboxylic acid (INCI name: Octadecenedioic Acid).

Particularly preferred are skin-lightening active substances e) from the group of ascorbic acid, 2-(4-hydroxyphenoxy)propionic acid, phenylethyl resorcinol, octadecenedioic acid, Olea europaea extract, ascorbyl tetraisopalmitate, magnesium ascorbyl phosphate, and ascorbyl glucoside.

As initially stated, the cosmetic compositions of the invention are suitable in a particular way to stabilize a rather high number of cosmetically active substances as well. A preferred subject for this reason constitutes cosmetic compositions that include at least two, preferably at least four, and in particular at least eight cosmetically active substances or active substance preparations.

Preferred cosmetic compositions include
at least two, preferably at least four organic UV filters, or
at least two, preferably at least four skin-lightening active substances, or
at least two, preferably at least four organic UV filters, at least two skin-lightening active substances, at least one vitamin, provitamin, or a compound designated as a vitamin precursor, as well as at least one plant extract different from the aforementioned active substances.

The compositions of the invention include as a further preferred optional component a linear alkane having 11 to 18 carbon atoms. Preferred are linear alkanes that have 11 to 13 carbon atoms, it being particularly preferred if the linear alkane has 11 and/or 13 carbon atoms.

The use of mixtures of undecane and tridecane is particularly preferred. Particularly preferred is the use of mixtures of undecane and tridecane in which the two linear alkanes are present in a weight ratio of 4:1 to 1:1, in particular of 3:1 to 1:1. Suitable substance mixtures are obtainable, for example, under the trade name Cetiol Ultimate.

It is not only the storage stability of the cosmetic compositions that is improved by the addition of the linear alkanes. Moreover, the addition of the above-described alkanes improves the solubility of oil-soluble UV filters, improves the spreadability of the composition on the skin, reduces the tackiness of the composition, and improves the skin feel after application of the composition to the skin.

Cosmetic compositions that include the linear alkane, based on the total weight of the compositions, in amounts of 0.1 to 10% by weight, preferably of 0.2 to 7.0% by weight, and in particular of 0.5 to 5.0% by weight are preferred.

### Overview Table

The composition of some preferred cosmetic compositions can be obtained from the following tables (data are given in percentages by weight, based on the total weight of the cosmetic composition, unless otherwise stated).

| | Formula 1 | Formula 2 | Formula 3 | Formula 4 |
|---|---|---|---|---|
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 5 | Formula 6 | Formula 7 | Formula 8 |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 11 | Formula 12 | Formula 13 | Formula 14 |
| Monoglycerol fatty | 0.01 to 6.0 | 0.1 to 4.0 | 0.1 to 4.0 | 0.5 to 2.0 |
| acid ester A | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 15 | Formula 16 | Formula 17 | Formula 18 |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I) | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 21 | Formula 22 | Formula 23 | Formula 24 |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 25 | Formula 26 | Formula 27 | Formula 28 |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 31 | Formula 32 | Formula 33 | Formula 34 |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 35 | Formula 36 | Formula 37 | Formula 38 |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 41 | Formula 42 | Formula 43 | Formula 44 |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 45 | Formula 46 | Formula 47 | Formula 48 |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 1a | Formula 2a | Formula 3a | Formula 4a |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 5a | Formula 6a | Formula 7a | Formula 8a |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 11a | Formula 12a | Formula 13a | Formula 14a |
| Monoglycerol fatty | 0.01 to 6.0 | 0.1 to 4.0 | 0.1 to 4.0 | 0.5 to 2.0 |
| acid ester A | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 15a | Formula 16a | Formula 17a | Formula 18a |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 21a | Formula 22a | Formula 23a | Formula 24a |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 25a | Formula 26a | Formula 27a | Formula 28a |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 31a | Formula 32a | Formula 33a | Formula 34a |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 35a | Formula 36a | Formula 37a | Formula 38a |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₄₋₁₈ fatty | 0.01 to 5.0 | 0.05 to 3.0 | 0.05 to 3.0 | 0.1 to 1.0 |
| alcohol | | | | |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 41a | Formula 42a | Formula 43a | Formula 44a |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl alcohol | 0.01 to 5.0 | 0.05 to 3.0 | 0.05 to 3.0 | 0.1 to 1.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 45a | Formula 46a | Formula 47a | Formula 48a |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl alcohol | 0.01 to 5.0 | 0.05 to 3.0 | 0.05 to 3.0 | 0.1 to 1.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 1c | Formula 2c | Formula 3c | Formula 4c |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 5c | Formula 6c | Formula 7c | Formula 8c |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 11c | Formula 12c | Formula 13c | Formula 14c |
| Monoglycerol fatty | 0.01 to 6.0 | 0.1 to 4.0 | 0.1 to 4.0 | 0.5 to 2.0 |
| acid ester A | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 15c | Formula 16c | Formula 17c | Formula 18c |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I) | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 21c | Formula 22c | Formula 23c | Formula 24c |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid esters of the | 0.05 to 3.0 | 0.1 to 2.0 | 0.1 to 2.0 | 0.2 to 1.5 |
| general formula (I), in | | | | |
| which the R₁and R₂ | | | | |
| groups independently | | | | |
| of one another stand | | | | |
| for an unsaturated C₁₆₋₂₆ | | | | |
| alkyl group | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 25c | Formula 26c | Formula 27c | Formula 28c |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 31c | Formula 32c | Formula 33c | Formula 34c |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 35c | Formula 36c | Formula 37c | Formula 38c |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 41c | Formula 42c | Formula 43c | Formula 44c |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Undecane, Tridecane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 45c | Formula 46c | Formula 47c | Formula 48c |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Undecane, Tridecane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 1d | Formula 2d | Formula 3d | Formula 4d |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 5d | Formula 6d | Formula 7d | Formula 8d |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 11d | Formula 12d | Formula 13d | Formula 14d |
| Monoglycerol fatty | 0.01 to 6.0 | 0.1 to 4.0 | 0.1 to 4.0 | 0.5 to 2.0 |
| acid ester A | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 15d | Formula 16d | Formula 17d | Formula 18d |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 21d | Formula 22d | Formula 23d | Formula 24d |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 25d | Formula 26d | Formula 27d | Formula 28d |
| Monoglycerol fatty | 0.01 to 6.0 | 0.1 to 4.0 | 0.1 to 4.0 | 0.5 to 2.0 |
| acid ester A | | | | |
| Polyglycerol di-fatty | 0.05 to 3.0 | 0.05 to 3.0 | 0.1 to 2.0 | 0.2 to 1.5 |
| acid ester B | | | | |
| Polyglycerol di-fatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 31d | Formula 32d | Formula 33d | Formula 34d |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 35d | Formula 36d | Formula 37d | Formula 38d |
| Mono-, di-, or triesters | 0.01 to 6.0 | 0.1 to 4.0 | 0.1 to 4.0 | 0.5 to 2.0 |
| of glycerol with | | | | |
| behenic acid | | | | |
| Di-, tri-, or | 0.05 to 3.0 | 0.05 to 3.0 | 0.1 to 2.0 | 0.2 to 1.5 |
| tetraglycerol di-fatty | | | | |
| acid esters of saturated | | | | |
| or unsaturated C₁₆₋₁₈ | | | | |
| fatty acids | | | | |
| Penta-, hexa-, or | 0.2 to 8.0 | 0.2 to 8.0 | 0.4 to 6.0 | 0.6 to 4.0 |
| heptaglycerol di-fatty | | | | |
| acid esters of saturated | | | | |
| or unsaturated C₁₆₋₁₈ | | | | |
| fatty acids | | | | |
| Fatty acid esters of the | 0.05 to 3.0 | 0.1 to 2.0 | 0.1 to 2.0 | 0.2 to 1.5 |
| general formula (I), in | | | | |
| which the R₁ and R₂ | | | | |
| groups independently | | | | |
| of one another stand | | | | |
| for an unsaturated C₁₆₋₂₆ | | | | |
| alkyl group | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |
| | Formula 41d | Formula 42d | Formula 43d | Formula 44d |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl alcohol | 0.01 to 5.0 | 0.05 to 3.0 | 0.05 to 3.0 | 0.1 to 1.0 |
| Undecane, Tridecane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance | | | | |
| Water | 30 to 82 | 30 to 82 | 35 to 75 | 45 to 60 |
| Misc. | To 100 | To 100 | To 100 | To 100 |
| | Formula 45d | Formula 46d | Formula 47d | Formula 48d |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl alcohol | 0.01 to 5.0 | 0.05 to 3.0 | 0.05 to 3.0 | 0.1 to 1.0 |
| Undecane, Tridecane | 0.1 to 10 | 0.2 to 7.0 | 0.2 to 7.0 | 0.5 to 5.0 |
| Thickener | 0.05 to 3.0 | 0.1 to 2.0 | 0.1 to 2.0 | 0.2 to 1.5 |
| Cosmetically active | 2.0 to 35 | 2.0 to 35 | 4.0 to 30 | 6.0 to 25 |
| substance ¹⁾ | | | | |
| Water | 30 to 82 | 30 to 82 | 35 to 75 | 45 to 60 |
| Misc. | To 100 | To 100 | To 100 | To 100 |

¹⁾ The cosmetically active substance is selected from the group of vitamins, provitamins, and vitamin precursors, plant extracts, organic UV filters, and skin-lightening active substances

As initially stated, a combination of the components a), b), c), and d) is suitable in a particular way for improving the physical and chemical stability of cosmetic UV protection agents. A further subject of the present invention is the use of a combination, comprising
a) at least one monoglycerol fatty acid ester A,
b) at least one first polyglycerol di-fatty acid ester B,
c) at least one second polyglycerol di-fatty acid ester C different from the first polyglycerol di-fatty acid ester B,
d) at least one fatty acid ester of the general formula (I)

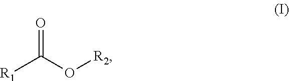

where the R₁ and R₂ groups independently of one another stand for a saturated or unsaturated alkyl groups having 16 to 26 carbon atoms,
to improve the storage stability of a cosmetic composition including at least one UV filter;
to improve the UV protective effect of a cosmetic composition including at least one UV filter;
to improve the absorption of a cosmetic composition including at least one UV filter;
to improve the spreadability and to reduce the tackiness of a cosmetic composition including at least one UV filter.

A third subject of the present invention lastly is a method for cosmetic skin care in which a cosmetic composition of the invention is applied to the skin, in particular to facial skin.

The statements made about the cosmetic agents of the invention apply mutatis mutandis to the use of the invention and the cosmetic method of the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition in the form of an oil-in-water emulsion, including:
a) at least one monoglycerol fatty acid ester A,
b) at least one first polyglycerol di-fatty acid ester B,
c) at least one second polyglycerol di-fatty acid ester C different from the first polyglycerol di-fatty acid ester B,
d) at least one fatty acid ester of the general formula (I) 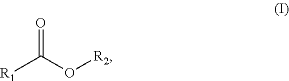
wherein the R₁ and R₂ groups independently of one another stand for a saturated or unsaturated alkyl groups having 16 to 26 carbon atoms,
e) at least one thickener, and
f) at least one cosmetically active substance.

2. The cosmetic composition according to claim 1, wherein the monoglycerol fatty acid ester A, based on the total weight of the compositions, is included in amounts of 0.01 to 6.0% by weight.

3. The cosmetic composition according to claim 1, wherein the first polyglycerol di-fatty acid ester B, based on the total weight of the compositions, is included in amounts of 0.05 to 3.0% by weight.

4. The cosmetic composition according to claim 1, wherein the second polyglycerol di-fatty acid ester C, based on the total weight of the compositions, is included in amounts of 0.2 to 8.0% by weight.

5. The cosmetic composition according to claim 1, wherein the thickener, based on the total weight of the compositions, is included in amounts of 0.05 to 3.0% by weight.

6. The cosmetic composition according to claim 1, wherein the cosmetically active substance, based on the total weight of the composition, is included in amounts of 2.0 to 35% by weight.

7. The cosmetic composition according to claim 1, wherein the composition includes furthermore
g) a linear fatty alcohol having 14 to 18 carbon atoms.

8. The cosmetic composition according to claim 1, wherein the composition includes furthermore
h) a linear alkane having 11 to 18 carbon atoms.

9. A method for cosmetic skin care including applying to the skin a cosmetic composition according to claim 1.
